(19) United States
(12) Patent Application Publication
(10) Pub. No.: US 2026/0034378 A1
(43) Pub. Date: Feb. 5, 2026

(54) ENHANCING PROTECTIVE EFFECTS IN TISSUES BY SELECTIVE APPLICATION OF MAGNETIC FIELDS

(71) Applicant: Robert E SANDSTROM, Longview, WA (US)

(72) Inventor: Robert E SANDSTROM, Longview, WA (US)

(21) Appl. No.: 18/791,865

(22) Filed: Aug. 1, 2024

(51) Int. Cl. A61N 2/00; A61N 2/02; A61N 2/06

(52) U.S. Cl. CPC A61N 2/004; A61N 2/002; A61N 2/02; A61N 2/06

(57) ABSTRACT

Methods of enhancing a protective effect in normal tissue adjacent to a target tissue, where the target tissue is a target for a therapeutic intervention that creates additional free radical entities within at least the target tissue. The methods include: Exposing the normal tissue to a magnetic field having a field strength sufficient to prolong a lifetime of a naturally-occurring free radical entity for a time sufficient to prolong the lifetime of the naturally-occurring free radical entity, and after exposing the normal tissue to the magnetic field, exposing the target tissue to the therapeutic intervention.

### TECHNICAL FIELD

The present disclosure relates to medical treatments, and more particularly to treatments including exposure to selective magnetic fields to trigger tissue responses that can provide enhanced tissue-protective effects.

### BACKGROUND

Free radical entities are be generated in tissue by several normal physiological and/or chemical events. A range of physiological mechanisms exist to maintain free radical physiological balance in living systems, including the presence of production of antioxidants, including enzymes, such as superoxide dismutase, catalases, glutathione, peroxidase, and other water-soluble and/or lipid-soluble antioxidants.

Free radical entities are also generated by irradiation of tissues. The study of high-intensity, short-duration, radiation therapies have provided evidence that normal (healthy) tissue exhibits a differential response to irradiation when compared to targeted tumor tissues. Specifically, in high-intensity radiation environments, normal tissue can exhibit subsequent enhanced resistance to radiation damage, as compared to radiated tumor tissue in the same biologic setting.

What is needed is a way to exploit the response of normal tissue to the presence of free radical entities in way that can confer a protective effect onto normal tissue with respect to subsequent therapeutic treatments.

### SUMMARY

The present disclosure is directed to methods of enhancing a protective effect in normal tissue adjacent to a target tissue, where the target tissue is a target for a therapeutic intervention that creates additional free radical entities within at least the target tissue. The methods include: Exposing the normal tissue to a magnetic field having a field strength sufficient to prolong a lifetime of a naturally-occurring free radical entity for a time sufficient to prolong the lifetime of the naturally-occurring free radical entity, and after exposing the normal tissue to the magnetic field, exposing the target tissue to the therapeutic intervention.

### DETAILED DESCRIPTION

The therapeutic ratio (or therapeutic index) is a comparison of the amount of a therapeutic agent that causes the desired therapeutic effect to the amount that causes toxicity. In some cases, the therapeutic ratio is a comparison of the damage done to normal tissue by the treatment versus the damage done to the targeted tissues, such as for example the tissue of a tumor. The therapeutic ratio is therefore one measure of the relative safety of a given therapy or treatment.

Various mechanisms have been offered to explain the observation of enhanced resistance to radiation damage by normal tissue after radiation treatment. Some researchers have identified transient hypoxic states induced in both tumor and adjacent normal tissues as one possible explanation for the relative enhanced radiation protection of normal tissue. Transient hypoxia in normal tissue is a stressor which can elicit protective tissue responses through enzymatic pathways, immune responses, and biochemical events. These responses differ from the response to transient hypoxia in tumor tissue, or other damaged or impaired tissues, which are frequently targeted by therapeutic interventions such as one or more of radiation therapy, chemo therapy, hyperthermia, or other targeted therapeutic efforts.

Hypoxia, as well as other physiological and biochemical stressors, can trigger, or are at least frequently associated with the release and/or production of free radicals in tissue. Free radicals by definition are chemical entities having a single unpaired electron in an outer molecular orbital. All free radicals, by virtue of having an unpaired electron, are subject to precession in the presence of an applied external magnetic field having a specific field strength.

The specific field strength that will influence the behavior of a free radical can be determined by the particular quantum eigenstate of that free radical entity, which is a discrete and specific energy state of that free radical entity. The application of a magnetic field of the appropriate specific discrete energy can cause the free radical entity to precess in a way that is comparable to Larmor presession of classical physics. This precession can preclude an immediate recombination of free radical pairs normally produced by the types of lytic events that can occur in a range of chemical and electrochemical reactions in normal tissues.

The presence of free radical entities, even if present in concentrations that are only marginally increased above normal physiological levels, can stimulate various biochemical and immune responses that will serve to mitigate the consequences of any non-physiological free radical creating events. Therefore, by the application of an external magnetic field having the appropriate and specific field strength, the recombination of those free radical entities already present at normal physiological levels can be delayed, increasing the concentration of free radical entities in the tissue, and thereby triggering the desirable protective effects associated with exposure to free radical entities without the necessity of radiation exposure or other potentially harmful treatments. Such tissues may then exhibit enhanced resistance to subsequent therapeutic treatments that might result in increased free radical concentration.

### Methods of Use

The present disclosure is directed to a method of enhancing a protective effect in normal tissues adjacent to a tumor, the method including the steps of exposing the normal tissues to a magnetic field having a field strength selected to prolong lifetimes of naturally-occurring free radical entities for a time sufficient to prolong the lifetimes of the naturally-occurring free radical entities, and after exposing the normal tissues to the magnetic field, exposing the tumor to a therapeutic treatment that creates additional free radical entities within at least the tumor.

For any of the techniques described herein, the magnetic field generated during magnetic field exposure can have a field strength of between 1-500 gauss. In one aspect of the disclosed methods, the strength of the applied magnetic field can be between 1-100 gauss. In another aspect of the disclosed methods, the strength of the applied magnetic field can be between 30-50 gauss.

If it is deemed desirable to limit or minimize the enhancement of free radical entity lifetimes in portions of the subject, or in selected tissues, magnetic field shielding devices can be employed during magnetic field exposure to shield such portions or tissues. The magnetic shielding devices can be provided as portable elements of the therapeutic magnetic field device, or devices that are worn by the patient during exposure. When used, the magnetic shielding devices may include one or more ferromagnetic metallic plates, such as for example an array of such ferromagnetic metallic plates. Alternatively, or in addition, a targeting of specific tissues can be accomplished by using a therapeutic magnetic field device that incorporates one or more movable source magnets, so that application of the therapeutic magnetic field device can be targeted to at least some extent.

The magnetic field used to enhance the protective effect in normal tissues can be generated by a therapeutic magnetic field device. A therapeutic magnetic field device typically includes a magnetic field generator, a magnetic field sensor, a data entry panel that permits a user to input time periods and electromagnetic field strengths for an electromagnetic field production regime, and a data processor including non-transitory computer readable memory, adapted to control said magnetic field generator to output a magnetic field in accordance with data received from said data entry panel.

The magnetic field can be generated by any appropriate magnetic field source, such as for example ferrimagnets or Helmholtz coils. In one aspect, the magnetic field sources are arrayed such that the magnetic field can be contoured to fit a desired topography of the tissue being exposed to the magnetic field. Alternatively, or in addition, one or more magnetic field sensors connected to the control station can be used to monitor the magnetic field in multiple axes. In one embodiment, the magnetic field sensors are Hall probes.

An engineer of ordinary background and education with regards to electromagnetic field generation devices can design and fabricate apparatus having electromagnetic coils that are capable of generating sufficiently uniform and/or contoured magnetic fields of specific field strength, as described herein, and which would be suitable for the methods described herein.

### CONCLUSION

The methods of treatment described in the present disclosure are able to create at least a marginally increased level of free radical entities within normal tissues, the presence of which triggers an early but significant physiologic response in that tissue that stimulates and enhances normal physiologic processes which are protective with respect to later, more severe free radical-related physiologic challenges. Tumor cells and tissues, and otherwise impaired tissues, necessarily respond less effectively to the initial enhanced low level free radical exposure, and therefore normal tissues are functionally less impaired and demonstrate longer survivals as opposed to impaired and tumor tissues.

More particularly, tissues that are targeted for therapeutic interventions, such as radiation therapy and/or chemotherapy, can be treated with a specific magnetic field of low strength, or a series of specific magnetic field strengths determined by the tissue being targeted for therapy and its biochemical environment. The applied specific magnetic field should be contoured to optimize the benefit to normal tissues, both adjacent to the tissue target to be irradiated or otherwise treated. In some instances, discrete anatomic, zones or organs and or portions of the body will be exposed to the low-level magnetic field. Magnetic field strengths will generally fall between 5 Gauss and 500 Gauss. Exposure times will vary from less than an hour to continuous exposure as may be dictated by clinical parameters. Field strengths are discrete, specific, and dictated by the quantum energy state of the free radical or radicals targeted. For normal tissues predominant free radicals can include singlet oxygen, superoxide anions, hydroxyl radicals, alkoxyl radicals, peroxyl radicals, hydrogen peroxide and lipid hydroperoxide.

### EXAMPLES

### Example 1

An adult male patient 41 years of age is to be treated for a right frontal lobe glioblastoma multiforme with photon radiation therapy. Immediately prior to each radiation treatment the patient's head and upper neck is given a continuous exposure to a 38 guass static magnetic field. The static magnetic field is applied using a helmet configured with an appropriate number and configuration of coils to produce a magnetic field of the appropriate strength in a region including the glioblastoma and adjacent healthy tissue. The patient is exposed to the static magnetic field for 30 minutes immediately prior to, or with one hour prior to, each radiation therapy fraction. The radiation treatment plan includes 35 daily fractions of 1.8 Gy applied to the bulk tumor and a 2-3 cm margin of adjacent brain tissue.

The therapeutic ratio of the radiation treatment after exposure to the magnetic field is determined, and then compared to the therapeutic ratio for the control radiation treatment without a prior magnetic field exposure. The determined therapeutic ratio for the radiation treatment following magnetic field exposure is lower than the therapeutic ratio determined for the radiation treatment in the absence of the prior exposure to the magnetic field.

### Example 2

An adult female patient 50 years of age with advanced metastatic breast carcinoma is to be treated with intravenous adriamycin at a dosage of 50 mg/m² body surface at 4 week intervals. For one hour prior to each intravenous dosage the patient is placed within a total body static magnetic of 38 gauss employing a system of electromagnetic coils that are configured to provide a substantially uniform magnetic field, producing a protective effect in the patient's healthy tissue.

Alternatively, the patient may be placed within a variable total body magnetic field, and the magnetic field strength is varied to create magnetic fields of between 10 gauss and 40 gauss. Field strength may be increased sequentially, and may be increased continuously or by incremental increase.

In the description and the claims, the term “substantially” means a deviation of up to 10% of the stated value, if physically possible, both downward and upward, otherwise only in the appropriate direction; in the case of degrees (angle and temperature), and for indications such as “parallel” or “normal,” this means±10°. For terms such as “substantially constant” etc., what is meant is the technical possibility of deviation which the person skilled in the art proceeds from, and not the mathematical one. For example, a “substantially L-shaped cross-section” comprises two elongated surfaces, which merge at one end into the end of the other surface, and whose longitudinal extensions are arranged at an angle of 45° to 120° to each other.

All given quantities and percentages, in particular those relating to the limitation of the invention, insofar as they do not relate to specific examples, are understood to have a tolerance of ±10%; accordingly, for example: 11% means 9.9% to 12.1%. With terms such as “a solvent,” the word “a” is not to be considered to represent a singular numeral, but rather is to be considered an indefinite article or pronoun, unless the context indicates otherwise.

The term: “combination” and/or “combinations,” unless otherwise stated, mean all types of combinations, starting from two of the relevant components up to a plurality or all of such components; the term “containing” also means “comprising.”

Although the present methods and apparatus have been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

Inventions embodied in various combinations and subcombinations of features, functions, elements, and/or properties may be claimed through presentation of new claims in related applications. Such new claims, whether they are directed to a different invention or directed to the same invention, whether different, broader, narrower or equal in scope to the original claims, are also regarded as included within the subject matter of the present disclosure.

What is claimed is:

1. A method of enhancing a protective effect in a normal tissue adjacent to a target tissue, where the target tissue is a target for a therapeutic intervention that creates additional free radical entities within at least the target tissue, comprising:
exposing the normal tissue to a magnetic field having a field strength sufficient to prolong a lifetime of a naturally-occurring free radical entity for a time sufficient to prolong the lifetime of the naturally-occurring free radical entity; and
after exposing the normal tissue to the magnetic field, exposing the target tissue to the therapeutic intervention.

2. The method of claim 1, wherein the therapeutic intervention is selected to treat infection, inflammation, or injury in the target tissue.

3. The method of claim 1, wherein the magnetic field is created by at least one magnet positioned exterior to the normal tissue.

4. The method of claim 1, wherein exposing the normal tissue to the magnetic field includes exposing the normal tissue to the magnetic field for between 5 minutes and 3 hours.

5. The method of claim 1, wherein exposing the normal tissue to the magnetic field includes exposing the normal tissue to the magnetic field for between 20 minutes and 2 hours.

6. The method of claim 1, wherein exposing the normal tissue to the magnetic field includes exposing the normal tissue to the magnetic field for between 40 minutes and 1.5 hours.

7. The method of claim 1, wherein exposing the normal tissue to the magnetic field includes exposing the normal tissue to a magnetic field having a field strength of between 1-500 gauss.

8. The method of claim 1, wherein exposing the normal tissue to the magnetic field includes exposing the normal tissue to a magnetic field having a field strength of between 1-100 gauss.

9. The method of claim 1, wherein exposing the normal tissue to the magnetic field includes exposing the normal tissue to a magnetic field having a field strength of between 30-50 gauss.

10. A method of enhancing a protective effect in a normal tissue adjacent to a tumor, comprising:
exposing the normal tissue to a magnetic field having a field strength selected to prolong a lifetime of a naturally-occurring free radical entity for a time sufficient to prolong the lifetime of the naturally-occurring free radical entity; and
after exposing the normal tissue to the magnetic field, exposing the tumor to a therapeutic treatment that creates additional free radical entities within at least the tumor.

11. The method of claim 10, wherein the therapeutic treatment includes a radiation treatment.

12. The method of claim 10, wherein the therapeutic treatment includes a chemotherapeutic treatment.

13. The method of claim 10, wherein the magnetic field is created by at least one magnet positioned exterior to the normal tissue.

14. The method of claim 10, wherein exposing the normal tissue to the magnetic field includes exposing the normal tissue to the magnetic field for between 5 minutes and 3 hours.

15. The method of claim 10, wherein exposing the normal tissue to the magnetic field includes exposing the normal tissue to the magnetic field for between 20 minutes and 2 hours.

16. The method of claim 10, wherein exposing the normal tissue to the magnetic field includes exposing the normal tissue to the magnetic field for between 40 minutes and 1.5 hours.

17. The method of claim 10, wherein exposing the normal tissue to the magnetic field includes exposing the normal tissue to a magnetic field having a field strength of between 1-500 gauss.

18. The method of claim 10, wherein exposing the normal tissue to the magnetic field includes exposing the normal tissue to a magnetic field having a field strength of between 1-100 gauss.

19. The method of claim 10, wherein exposing the normal tissue to the magnetic field includes exposing the normal tissue to a magnetic field having a field strength of between 30-50 gauss.
